(19) United States
(12) Patent Application Publication
(10) Pub. No.: US 2025/0304696 A1
(43) Pub. Date: Oct. 2, 2025

(54) METHODS AND COMPOSITIONS FOR TREATING BARTH SYNDROME

(71) Applicants: PROVENTION BIO, INC., RED BANK, NJ (US); UNIVERSITY OF FLORIDA RESEARCH FOUNDATION, INCORPORATED, GAINESVILLE, FL (US)

(72) Inventors: FRANCISCO LEON, RED BANK, NJ (US); PAUL DUNFORD, RED BANK, NJ (US); DIANA MILLER, RED BANK, NJ (US); BARRY JOHN BYRNE, GAINESVILLE, FL (US); MANUELA CORTI, GAINESVILLE, FL (US)

(21) Appl. No.: 18/722,261

(22) PCT Filed: Dec. 23, 2022
(86) PCT No.: PCT/US2022/082367

§ 371 Date: Jun. 20, 2024

Related U.S. Application Data
(60) Provisional application No. 63/293,514, filed on Dec. 23, 2021.

(51) Int. Cl. C07K 16/28; A61K 31/436; A61K 38/17; A61K 48/00; A61P 37/02

(52) U.S. Cl. CPC C07K 16/283; A61K 31/436; A61K 38/17; A61K 48/005; A61P 37/02; C07K 16/2896; C07K 2317/31; C07K 2317/56; C07K 2317/626; C07K 2317/76

(57) ABSTRACT

Disclosed herein, in one aspect, is a method of reducing immunogenicity, comprising administering to a patient receiving or having received a BTHS gene therapy, an effective amount of B cell inhibitor that is non-depletional. Related compositions are also provided.

### CROSS REFERENCE TO RELATED APPLICATIONS

This application is a U.S. National Stage application of PCT/US2022/082367 filed on 23 Dec. 2022, which claims benefit of and priority to U.S. Provisional Patent Application No. 63/293,514 filed Dec. 23, 2021, the contents of which is incorporated herein in its entirety.

### SEQUENCE LISTING

This specification contains a Sequence Listing, which has been submitted electronically in XML format and is hereby incorporated by reference in its entirety. Said XML copy, created on 12 Jan. 2025, is named 0171_0112-PCT-US_Sequence Listing.xml and is 19,760 bytes in size.

### FIELD

The present disclosure generally relates to compositions and methods for reducing immunogenicity of biological therapeutics, and more particularly in connection with gene therapy for the treatment of Barth Syndrome.

### BACKGROUND

Barth Syndrome (OMIM #302060; BTHS), first discovered in 1983 by Dr. Peter Barth, is a rare X-linked genetic disorder caused by a pathologic variation in the TAFAZZIN gene that encodes an acyltransferase necessary for remodeling of cardiolipin. Mutations in the TAFAZZIN gene, located at Xq28, therefore cause pathologic features by leading to accumulation of intermediates of cardiolipin metabolism and mitochondrial dysfunction. The effects are varied in presentation and severity, in part due to differences in tissue specific gene expression. Clinical pathology typically includes cardiovascular dysfunction, myopathy, and immunodeficiency predisposing to infections. Mutations leading to BTHS are typically familial but 13% are estimated to be de novo (Gonzalez 2012). There are estimated to be approximately 150-200 known individuals with BTHS who are living at present. However, it is becoming increasingly recognized that the numbers of individuals living with this condition undiagnosed may be greater, with 1 in 140,000 lives births being affected.

Cardiomyopathy (CM) is estimated to occur in up to 70% of individuals with BTHS and presents within the first year of life. Cardiovascular pathology includes dilated CM, left ventricular non-compaction, hypertrophic CM, and rarely QTc prolongation, arrythmias, and sudden cardiac death. Delays in diagnosis of CM in BTHS are common and individuals require careful monitoring by cardiologists for heart failure. Fortunately, most patients with CM respond well to standard medical therapy. However, a proportion of patients with BTHS progress to require heart transplantation within the first 5 years of life. In addition, it has been noted in larger cohort studies that ventricular arrythmias are common and >10% of patients require placement of an implantable defibrillator due to risk of fatal arrythmias. Arrythmias can occur in individuals with BTHS during periods of otherwise seemingly good health.

Immunodeficiencies which predispose to serious infections or septicemia are common in BTHS. Most common is neutropenia which occurs in up to 90% of patients. The neutropenia can range from mild and episodic to severe and chronic. In addition, pancytopenia has been noted in individuals with BTHS and often mistaken for viral-induced bone marrow suppression (Rigaud 2013). It should be noted that half of the patients described with early demise in Barth's original paper succumbed to complications of infection (Barth 1983). The leading hypothesis to the cause of neutropenia is that there are dysfunctional neutrophil precursors in the bone marrow. The use of granulocyte colony stimulating factor (G-CSF) is often used to ameliorate low neutrophil counts, however infections are still commonplace despite this.

Neuromuscular effects are common. Skeletal myopathy includes proximal, non-progressive muscle weakness. Hypotonia in childhood leads to gross motor developmental delays and in adulthood leads to easy fatiguability and reduced quality of life. Clinical trials aimed at supporting aerobic training seemed to improve quality of life but had little measurable physiological benefit.

Measurable metabolic abnormalities are helpful in making diagnosis of BTHS with 5 to 20-fold increase in urinary 3-methylglutaconic acid. True metabolic sequelae are rare but lactic acidosis and hypoglycemia are more common in infancy and can be fatal. Current therapy is aimed at supportive care during acute illness.

Advances in standard medical care have made BTHS a survivable condition into adulthood. Currently recommended therapies for BTHS are supportive but not curative. Supplements such as coenzyme Q, carnitine, pantothenic acid and other B vitamins typically used to treat other mitochondrial diseases have not proven effective in BTHS (Rugolotto 2003).

However, there is significant variability in presentation and when individuals fall on the severe spectrum of disease, the complications can be life-threatening requiring cardiac transplantation or extracorporeal membrane oxygenation (ECMO) to survive. Even in more mild or moderate cases of BTHS, there is risk of acute metabolic crisis or severe infection that present with little to no warning (barthsyndrome.org). This is a significant source of anxiety for many patients who survive into adulthood.

BTHS patients endure lifelong limitations and reduced quality of life from both muscle weakness and chronic fatigue. Clinical trials utilizing formal exercise programs to build strength and endurance have proven ineffective, as have a myriad of supplements. Thus, there is an unmet need in the care for BTHS patients. A panel of patients met for the first time with the FDA in 2018 to help explain their concerns and advocate for therapies beyond mere symptom management.

Advances afforded by the burgeoning therapeutic field of gene therapy are quite appealing for the treatment of Barth Syndrome. Gene therapy allows restoration of the normal production of acyltransferase thereby reducing clinical symptomatology.

In gene therapy, genetically modified cells are generated for the purpose of incorporating a missing copy of a gene. However, there is a risk that some of the “machinery” utilized for the genetic modification of the cells could be “presented” by the modified cells and be recognized by the host as a “foreign” agent. Such recognition would trigger a rejection reaction, which could potentially render ineffective such treatments or, in severe cases, potentially cause auto-immune reactions.

More recently, the advent of genetic therapies has seen the substantial obstacle of immunogenicity of the viral vector utilized to administer the transgene, as well as the immunogenicity of the transgene protein itself after expression by the recipient's cells. The immunogenicity of vectors and transgenes results in: 1) diminished efficacy as vector and transgene are bound and cleared by the antibodies generated by the recipient; 2) need for increased doses, which increase safety risks and costs; 3) difficulty or impossibility to re-dose if the subject develops antibodies against the vector or transgene after a prior dose. Sometimes, the recipients have pre-existing antibodies against the vector even before the first administration, due to cross-reaction with naturally-occurring viruses.

As such, a need exists for methods and compositions for reducing immunogenicity induced by Barth Syndrome gene therapy.

### SUMMARY

Disclosed herein, in one aspect, is a method of reducing immunogenicity associated with gene therapy for Barth Syndrome (BTHS), comprising administering to a patient receiving or having received a BTHS gene therapy, an effective amount of B cell inhibitor that is non-depletional.

In some embodiments, the BTHS gene therapy can include a recombinant adeno-associated virus (rAAV) vector (such as AAV9 vector) encoding a TAFAZZIN transgene.

In some embodiments, the B cell inhibitor is a CD32B×CD79B bi-specific antibody capable of immunospecifically binding an epitope of CD32B and an epitope of CD79B. In some embodiments, the CD32B×CD79B bi-specific antibody comprises:
(A) a VL_{CD32B} domain that comprises the amino acid sequence of SEQ ID NO: 1;
(B) a VH_{CD32B} domain that comprises the amino acid sequence of SEQ ID NO: 2;
(C) a VL_{CD79B} domain that comprises the amino acid sequence of SEQ ID NO: 3; and
(D) a VH_{CD79B} domain that comprises the amino acid sequence of SEQ ID NO: 4.

In some embodiments, the CD32B×CD79B bi-specific antibody is an Fc diabody comprising:
(A) a first polypeptide chain that comprises the amino acid sequence of SEQ ID NO: 5;
(B) a second polypeptide chain that comprises the amino acid sequence of SEQ ID NO: 6; and
(C) a third polypeptide chain that comprises the amino acid sequence of SEQ ID NO: 7.

In some embodiments, the method can further include administering the Fc diabody at a dose of between about 5 mg/kg and about 100 mg/kg, or between about 5 mg/kg and about 50 mg/kg, or between about 5 mg/kg and about 40 mg/kg and at a dosage regimen of between one dose per week and one dose per 6 weeks. In some embodiments, the method can include administering the Fc diabody at a dose of about 10 mg/kg, and at a dosage regimen of one dose per 1-4 weeks. In some embodiments, the method can include administering 3 doses of the Fc diabody at a dose of about 10 mg/kg at 2-6 week intervals.

One exemplary dosing regimen includes a first dose at 1 week prior to a first BTHS gene therapy delivery, a second dose at 2 weeks after the first BTHS gene therapy delivery, and a third dose 3-4 weeks following a second BTHS gene therapy delivery.

In some embodiments, the method can include administering a first dose about 2 days to about 6 weeks (e.g., 2 days, 6 days, 1 week, 2 weeks, 4 weeks) prior to administration of the BTHS gene therapy, a second dose at about the same time as administration of the BTHS gene therapy, and a third dose about 2 days to about 6 weeks (e.g., 2 days, 6 days, 1 week, 2 weeks, 4 weeks) after administration of the BTHS gene therapy.

In some embodiments, the Fc diabody results in inhibition of its own immunogenicity upon administration, with lower prevalence and/or titers of anti-drug antibodies (ADA) at increased doses. In some embodiments, the ADA does not neutralize the Fc diabody.

In some embodiments, the Fc diabody, in a dose-dependent fashion, binds to at least 80% of the B cells upon administration, and remains bound to at least 50% of the B cells for at least 4 weeks after last administration.

In some embodiments, the Fc diabody results in sustained inhibition of immunoglobulin production without depleting circulating B cells. In some embodiments, the immunoglobulins include one or more of IgM, IgA, IgG and IgE.

In some embodiments, the method can further include monitoring the patient by examining the presence of specific antibodies against the BTHS gene therapy. In some embodiments, the method can further include administering one or more dose of the B cell inhibitor to further modulate immunogenicity.

In some embodiments, the method can further include co-administering one or more immune-modulators, such as sirolimus, rapamycin, abatacept, teplizumab and immunoglobulin G-degrading enzyme of Streptococcus pyogenes. In some embodiments, the method can further include co-administering sirolimus.

Also provided herein are pharmaceutical compositions comprising the non-depletional B cell inhibitors disclosed herein, provided (e.g., packaged) at therapeutically effective unit doses. Instructions for dosage regimens as disclosed herein can also be provided.

A further aspect relates to use of the B cell inhibitor that is non-depletional as disclosed herein, in the manufacture of a medicament for reducing immunogenicity associated with Barth Syndrome (BTHS) gene therapy, wherein optionally the BTHS gene therapy comprises genetically modified cells having a recombinant adeno-associated virus (rAAV) vector encoding a TAFAZZIN transgene.

Also provided herein is a pharmaceutical composition for reducing immunogenicity associated with gene therapy for Barth Syndrome (BTHS), comprising an effective amount of a CD32B×CD79B bi-specific antibody capable of immunospecifically binding an epitope of CD32B and an epitope of CD79B, prior to, concurrently with, and/or after BTHS gene therapy, wherein optionally the BTHS gene therapy comprises genetically modified cells having a recombinant adeno-associated virus (rAAV) vector encoding a TAFAZZIN transgene.

The CD32B×CD79B bi-specific antibody can, in some embodiments, comprise:
(A) a VL_{CD32B} domain that comprises the amino acid sequence of SEQ ID NO: 1;
(B) a VH_{CD32B} domain that comprises the amino acid sequence of SEQ ID NO: 2;
(C) a VL_{CD79B} domain that comprises the amino acid sequence of SEQ ID NO: 3; and
(D) a VH_{CD79B} domain that comprises the amino acid sequence of SEQ ID NO: 4.

The CD32B×CD79B bi-specific antibody can be an Fc diabody comprising:
(A) a first polypeptide chain that comprises the amino acid sequence of SEQ ID NO: 5;
(B) a second polypeptide chain that comprises the amino acid sequence of SEQ ID NO: 6; and
(C) a third polypeptide chain that comprises the amino acid sequence of SEQ ID NO: 7.

The pharmaceutical composition, in some embodiments, can comprise the Fc diabody at a dose of between about 5 mg/kg and about 100 mg/kg, or between about 5 mg/kg and about 50 mg/kg, or between about 5 mg/kg and about 40 mg/kg, and at a dosage regimen of between one dose per week and one dose per 6 weeks.

The pharmaceutical composition, in some embodiments, can comprise the Fc diabody at a dose of about 10 mg/kg, and at 2-6 week intervals, or at one dose per 1-4 weeks. In certain embodiments, the dosing regimen can include a first dose at 1 week prior to a first BTHS gene therapy delivery, a second dose at 2 weeks after the first BTHS gene therapy delivery, and a third dose at 3-4 weeks following a second BTHS gene therapy delivery.

The pharmaceutical composition, in some embodiments, can comprise a first dose about 2 days to about 6 weeks prior to administration of the BTHS gene therapy, a second dose at about the same time as administration of the BTHS gene therapy, and a third dose about 2 days to about 6 weeks after administration of the BTHS gene therapy.

The pharmaceutical composition, in some embodiments, can further comprise one or more immune-modulators selected from sirolimus, rapamycin, abatacept, teplizumab and immunoglobulin G-degrading enzyme of Streptococcus pyogenes.

### BRIEF DESCRIPTION OF THE DRAWINGS

FIG. 1 is a schematic overview of an exemplary vector.

FIG. 2 is an exemplary construct B951 (pds2TR-Des-coTAZ_Dual4).

FIG. 3 is an exemplary construct C064 (pds2TR-Des-coTAZiso2_Dual4).

### DETAILED DESCRIPTION

Disclosed herein, in one aspect, is a method of reducing immunogenicity associated with gene therapy for Barth Syndrome (BTHS), comprising administering to a patient receiving or having received a BTHS gene therapy, an effective amount of B cell inhibitor that is non-depletional. In some embodiments, the B cell inhibitor is a CD32B×CD79B bi-specific antibody such as those disclosed in U.S. Publication No. 2016/0194396, WIPO Publication Nos. WO 2015/021089 and WO 2017/214096, each incorporated by reference in its entirety.

### Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value, or the variation that exists among the study subjects. Typically, the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The term “substantially” means more than 50%, preferably more than 80%, and most preferably more than 90% or 95%.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps. It is contemplated that any embodiment discussed in this specification can be implemented with respect to any method, system, host cells, expression vectors, and/or composition of the invention. Furthermore, compositions, systems, host cells, and/or vectors of the invention can be used to achieve methods and proteins of the invention.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

The use of the term “for example” and its corresponding abbreviation “e.g.” (whether italicized or not) means that the specific terms recited are representative examples and embodiments of the invention that are not intended to be limited to the specific examples referenced or cited unless explicitly stated otherwise.

A “gene” refers to an assembly of nucleotides that encode a polypeptide, and includes cDNA and genomic DNA nucleic acid molecules. “Gene” also refers to a nucleic acid fragment that can act as a regulatory sequences preceding (5′ non-coding sequences) and following (3′ non-coding sequences) the coding sequence.

As used herein, “TAFAZZIN” refers to a phospholipid-lysophospholipid transacylase that can be responsible for modification of cardiolipin (a membrane phospholipid) to its tetralinoleoyl form. In some embodiments, TAFAZZIN can refer to full-length human TAFAZZIN or human TAFAZZIN lacking exon 5, both of which can exhibit transacylase activity. In certain embodiments, tafazzin can refer to full-length mouse TAFAZZIN, which is homologous to the human TAFAZZIN lacking exon 5.

“Antibody” or “antibody molecule” as used herein refers to a protein, e.g., an immunoglobulin chain or fragment thereof, comprising at least one immunoglobulin variable domain sequence. An antibody molecule encompasses antibodies (e.g., full-length antibodies) and antibody fragments. In some embodiments, an antibody molecule comprises an antigen binding or functional fragment of a full-length antibody, or a full-length immunoglobulin chain. For example, a full-length antibody is an immunoglobulin (Ig) molecule (e.g., IgG) that is naturally occurring or formed by normal immunoglobulin gene fragment recombinatorial processes). In some embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)₂ fragments, and single chain variable fragments (scFvs). The terms “Fab” and “Fab fragment” are used interchangeably and refer to a region that includes one constant and one variable domain from each heavy and light chain of the antibody, i.e., V_{L}, C_{L}, V_{H}, and C_{H}1.

Throughout the present specification, the numbering of the residues in the constant region of an IgG Heavy Chain is that of the EU index as in Kabat et al., Sequences of Proteins of Immunological Interest, 5^{th} Ed. Public Health Service, NH1, MD (1991) (“Kabat”), expressly incorporated herein by references. The term “EU index as in Kabat” refers to the numbering of the human IgG1 EU antibody. Amino acids from the Variable Domains of the mature heavy and Light Chains of immunoglobulins are designated by the position of an amino acid in the chain. Kabat described numerous amino acid sequences for antibodies, identified an amino acid consensus sequence for each subgroup, and assigned a residue number to each amino acid, and the CDRs are identified as defined by Kabat (it will be understood that CDR_{H}1 as defined by Chothia, C. & Lesk, A. M. ((1987) “Canonical structures for the hypervariable regions of immunoglobulins,”. J. Mol. Biol. 196:901-917) begins five residues earlier). Kabat's numbering scheme is extendible to antibodies not included in his compendium by aligning the antibody in question with one of the consensus sequences in Kabat by reference to conserved amino acids. This method for assigning residue numbers has become standard in the field and readily identifies amino acids at equivalent positions in different antibodies, including chimeric or humanized variants. For example, an amino acid at position 50 of a human antibody Light Chain occupies the equivalent position to an amino acid at position 50 of a mouse antibody Light Chain.

In some embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule.

The terms “bispecific antibody molecule,” “diabody” and “Dual Affinity Re-Targeting (DART®)” antibody are used interchangeably herein and refer to an antibody molecule that has specificity for more than one (e.g., two, three, four, or more) epitope and/or antigen. In some embodiments, the antibody can be diabodies or scaffolds capable of antigen binding, such as those disclosed in U.S. Publication No. 2016/0194396, WIPO Publication Nos. WO 2015/021089 and WO 2017/214096, each incorporated by reference in its entirety. In some embodiments, the antibody can be CD32B×CD79B bispecific diabodies (i.e., “CD32B×CD79B diabodies,” and such diabodies that additionally comprise an Fc domain (i.e., “CD32B×CD79B Fc diabodies”). In some embodiments, the antibody can be a humanized CD32B×CD79B DART® antibody, produced in Chinese hamster ovary cells with a molecular weight of 111.5 kDa.

“Antigen” (Ag) as used herein refers to a macromolecule, including all proteins or peptides. In some embodiments, an antigen is a molecule that can provoke an immune response, e.g., involving activation of certain immune cells and/or antibody generation. Antigens are not only involved in antibody generation. T cell receptors also recognized antigens (albeit antigens whose peptides or peptide fragments are complexed with an MHC molecule). Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In some embodiments, an antigen does not need to be encoded solely by a full length nucleotide sequence of a gene, nor does an antigen need to be encoded by a gene at all. In some embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The “antigen-binding site” or “antigen-binding fragment” or “antigen-binding portion” (used interchangeably herein) of an antibody molecule refers to the part of an antibody molecule, e.g., an immunoglobulin (Ig) molecule such as IgG, that participates in antigen binding. In some embodiments, the antigen-binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions” (FRs). FRs are amino acid sequences that are naturally found between, and adjacent to, hypervariable regions in immunoglobulins. In some embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4. Variable light chain (VL) CDRs are generally defined to include residues at positions 27-32 (CDR1), 50-56 (CDR2), and 91-97 (CDR3). Variable heavy chain (VH) CDRs are generally defined to include residues at positions 27-33 (CDR1), 52-56 (CDR2), and 95-102 (CDR3). One of ordinary skill in the art would understand that the loops can be of different length across antibodies and the numbering systems such as the Kabat or Chothia control so that the frameworks have consistent numbering across antibodies.

In some embodiments, the antigen-binding fragment of an antibody (e.g., when included as part of a fusion molecule) can lack or be free of a full Fc domain. In some embodiments, an antibody-binding fragment does not include a full IgG or a full Fc but may include one or more constant regions (or fragments thereof) from the light and/or heavy chains. In some embodiments, the antigen-binding fragment can be completely free of any Fc domain. In some embodiments, the antigen-binding fragment can be substantially free of a full Fc domain. In some embodiments, the antigen-binding fragment can include a portion of a full Fc domain (e.g., CH2 or CH3 domain or a portion thereof). In some embodiments, the antigen-binding fragment can include a full Fc domain. In some embodiments, the Fc domain is an IgG domain, e.g., an IgG1, IgG2, IgG3, or IgG4 Fc domain. In some embodiments, the Fc domain comprises a CH2 domain and a CH3 domain.

As used herein, “administering” and similar terms mean delivering the composition to an individual being treated. Preferably, the compositions of the present disclosure are administered by, e.g., parenteral, including subcutaneous, intramuscular, or preferably intravenous routes.

As used herein, an “effective amount” means the amount of bioactive agent or diagnostic agent that is sufficient to provide the desired local or systemic effect at a reasonable risk/benefit ratio as would attend any medical treatment or diagnostic test. This will vary depending on the patient, the disease, the treatment being effected, and the nature of the agent. A therapeutically effective amount will vary depending upon the patient and disease condition being treated, the weight and age of the patient, the severity of the disease condition, the manner of administration and the like, which can readily be determined by one of ordinary skill in the art. The dosages for administration can range from, for example, about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg of an antibody or antigen binding portion thereof, as provided herein. Dosing may be, e.g., every week, every 2 weeks, every three weeks, every 4 weeks, every 5 weeks or every 6 weeks. Dosage regimens may be adjusted to provide the optimum therapeutic response. An effective amount is also one in which any toxic or detrimental effects (side effects) of the agent are minimized and/or outweighed by the beneficial effects. Administration may be intravenous at exactly or about 6 mg/kg or 12 mg/kg weekly, or 12 mg/kg or 24 mg/kg biweekly. Additional dosing regimens are described below.

As used herein, “pharmaceutically acceptable” shall refer to that which is useful in preparing a pharmaceutical composition that is generally safe, non-toxic, and neither biologically nor otherwise undesirable and includes that which is acceptable for veterinary use as well as human pharmaceutical use. Examples of “pharmaceutically acceptable liquid carriers” include water and organic solvents. Preferred pharmaceutically acceptable aqueous liquids include PBS, saline, and dextrose solutions etc.

The term “immunogenicity” refers to the ability of a particular substance, such as an antigen or epitope, to provoke an immune response, which can be humoral and/or cell-mediated, in the body of a human and other animal. In some embodiments, administration of the composition of the present disclosure reduces the immunogenicity of, and/or increases the immune tolerance to, a biological substance such as therapeutics. “Tolerance” or “immune tolerant” as used herein, refers to the absence of an immune response to a specific antigen (e.g., the therapeutic biologic) in the setting of an otherwise substantially normal immune system.

A “major histocompatibility complex” or “MHC” protein as used herein refers to a set of cell surface molecules encoded by a large gene family that play a significant role in the immune system of vertebrates. A key function of these proteins is to bind peptide fragments derived from endogenous or exogenous (foreign) proteins and display them on the cell surface for recognition by the appropriate T-cells of the host organism. The MHC gene family is divided into three subgroups: Class I, Class II, and Class III. The human MHC Class I and Class II genes are also referred to as human leukocyte antigen (HLA)—HLA Class I and HLA Class II, respectively. Some of the most studied HLA genes in humans are the nine MHC genes: HLA-A, HLA-B, HLA-C, HLA-DPAl, HLA-DPBl, HLA-DQAl, HLA-DQBl, HLA-DRA, HLA-DRBl and HLA-DRB345.

Various aspects of the disclosure are described in further detail below. Additional definitions are set out throughout the specification.

### Non-Depleting B Cell Inhibitors and Pharmaceutical Compositions

In some embodiments, a B cell inhibitor can be used to reduce or modulate immunogenicity. In some embodiments, such B cell inhibitors are non-depletional immunomodulators. As used herein, “non-depletional” or “non-depleting” means that the inhibitor or immunomodulator does not completely deplete B cell activities. On the other hand, “depletion” of B cells means that the agent acts to eliminate or destroy B cells, such as anti-CD20 antibodies, e.g., Rituximab. Thus, in some embodiments, the non-depletional B cell inhibitors or immunomodulators disclosed herein are not Rituximab. In some embodiments, the non-depletional B cell inhibitors or immunomodulators are not anti-CD20 antibodies or other CD20 inhibitors.

Exemplary non-depletional B cell inhibitors include, but are not limited to, CD32B×CD79B bi-specific inhibitors; CD32B modulators; B cell receptor (BCR) blockers, e.g., anti-CD22 molecules; B cell survival and activation inhibitors, e.g., B-cell activating factor (BAFF) or A proliferation-inducing ligand (APRIL) inhibitors such as belimumanb; anti-CD40 and anti-CD40L molecules; and Bruton's tyrosine kinase (BTK) inhibitors such as Ibrutinib (PCI-32765) and Acalabrutinib.

In some embodiments, the B cell inhibitor can be a CD32B×CD79B bi-specific antibody such as those disclosed in U.S. Publication No. 2016/0194396, WIPO Publication Nos. WO 2015/021089, and WO 2017/214096, all incorporated by reference in its entirety, or an antigen-binding fragment thereof.

An exemplary CD32B×CD79B bispecific diabody can comprise two or more polypeptide chains, and can comprise:

(1) a VL Domain of an antibody that binds CD32B
(VL_{CD32B}), such VL_{CD32B} Domain having the
sequence (SEQ ID NO: 1):
DIQMTQSPSS LSASμGDRVT ITCRASQEIS GYLSWLQQKP
GKAPRRLIYA ASTLDSGVPS RFSGSESGTE FTLTISSLQP
EDFATYYCLQ YFSYPLTFGG GTKVEIK
(2) A VH Domain of an antibody that binds CD32B
(VH_{CD32B}), such VH_{CD32B} Domain having
the sequence (SEQ ID NO: 2):
EVQLVESGGG LVQPGGSLRL SCAASGFTFS DAWMDWVRQA
PGKGLEWVAE IRNKAKNHAT YYAESVIGRF TISRDDAKNS
LYLQMNSLRA EDTAVYYCGA LGLDYWGQGT LVTVSS
(3) A VL Domain of an antibody that binds CD79B
(VL_{CD79B}), such VL_{CD79B} Domain having
the sequence (SEQ ID NO: 3):
DVVMTQSPLS LPVTLGQPAS ISCKSSQSLL DSDGKTYLNW
FQQRPGQSPN RLIYLVSKLD SGVPDRFSGS GSGTDFTLKI
SRVEAEDμGV YYCWQGTHFP LTFGGGTKLE IK
(4) A VH Domain of an antibody that binds CD79B
(VH_{CD79B}), such VH_{CD79B} Domain having
the sequence (SEQ ID NO: 4):
QVQLVQSGAE VKKPGASVKV SCKASGYTFT SYWMNWVRQA
PGQGLEWIGM IDPSDSETHY NQKFKDRVTM TTDTSTSTAY
MELRSLRSDD TAVYYCARAM GYWGQGTTVT VSS

In some embodiments, the B cell inhibitor can be PRV-3279, a humanized CD32B×CD79B Dual Affinity Re-Targeting (DART®) protein produced in Chinese hamster ovary cells with a molecular weight of 111.5 kDa. DART® proteins are bispecific, antibody-based molecules that can bind 2 distinct antigens simultaneously. PRV-3279 is designed to target CD32B (Fc gamma receptor IIb) and CD79B (immunoglobulin-associated beta subunit of the B cell receptor (BCR) complex) on B lymphocytes. Co-ligation of CD32B and CD79B in preferential cis-binding mode on B lymphocytes triggers CD32B-coupled immunoreceptor tyrosine-based inhibitory motif signaling, which decreases antigen-mediated naïve and memory B cell activation without broad depletion. To prolong in vivo half-life, PRV-3279 also contains a human immunoglobulin G (IgG)1 Fc region that has been mutated to greatly reduce or eliminate undesired binding to FcγTRs and complement but retains affinity for the neonatal FcR binding to take advantage of the IgG salvage pathway mediated by this receptor.

The CD32B molecule is a transmembrane inhibitory receptor expressed widely on B cells and other immune effector cells such as macrophages, neutrophils, and mast cells. The anti-CD32B component of PRV-3279 is based on a humanized version of MacroGenics' proprietary murine monoclonal antibody (mAb) 8B5. CD79B is an essential signal transduction component of the BCR that is expressed exclusively on B cells. The anti-CD79B component of PRV-3279 is based on a humanized version of the murine mAb CB3.

In some embodiments, PRV-3279 comprises the following sequence (the CDRs are underlined and coil domains are in bold):

Chain1 (Fc - CD32BVL - CD79bVH - E coil):
(SEQ ID NO.: 5)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLWCLVK
GFYPSDIAVEWESNGQPENNYKTTPPVLDSDGSFFLYSKLTVDKSRWQQG
NVFSCSVMHEALHNHYTQKSLSLSPGKAPSSSPMEDIQMTQSPSSLSASμ
GDRVTITCRASQEISGYLSWLQQKPGKAPRRLIYAASTLDSGVPSRFSGS
ESGTEFTLTISSLQPEDFATYYCLQYFSYPLTFGGGTKVEIKGGGSGGGG
QVQLVQSGAEVKKPGASVKVSCKASGYTFTSYWMNWVRQAPGQGLEWIGM
IDPSDSETHYNQKFKDRVTMTTDTSTSTAYMELRSLRSDDTAVYYCARAM
GYWGQGTTVTVSSGGCGGGEVAALEKEVAALEKEVAALEKEVAALEKGGG
NS
Chain2 (CD79bVL - CD32BVH - K coil):
(SEQ ID NO.: 6)
DVVMTQSPLSLPVTLGQPASISCKSSQSLLDSDGKTYLNWFQQRPGQSPN
RLIYLVSKLDSGVPDRFSGSGSGTDFTLKISRVEAEDμGVYYCWQGTHFP
LTFGGGTKLEIKGGGSGGGGEVQLVESGGGLVQPGGSLRLSCAASGFTFS
DAWMDWVRQAPGKGLEWVAEIRNKAKNHATYYAESVIGRFTISRDDAKNS
LYLQMNSLRAEDTAVYYCGALGLDYWGQGTLVTVSSGGCGGGKVAALKEK
VAALKEKVAALKEKVAALKE
Chain3 (Fc):
(SEQ ID NO.: 7)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLSCAVK
GFYPSDIAVEWESNGQPENNYKTTPPVLDSDGSFFLVSKLTVDKSRWQQG
NVFSCSVMHEALHNRYTQKSLSLSPGK

PRV-3279 binds to most B cells (e.g., >80-90%) in a dose-dependent fashion, including both naive and memory phenotypes upon administration and can remain bound to at least 50% of the B cells for more than 4 weeks. This shows sustained durability of the potential pharmacodynamic (PD) effect of PRV-3279 and supports a dosing frequency of once every month (or longer) administration.

PRV-3279 has previously been shown to reduce immunity to a viral vaccine (Hepatitis A vaccine) in healthy subjects, suggesting that immunogenicity to viral vectors can be inhibited. In addition, a pilot study was performed with the purpose to evaluate the effect of PRV-3279 surrogate on gene therapy using an adeno-associated virus (AAV) to assess the efficacy and immunogenicity in huCD32b transgenic mice (see PCT/US2020/044312 and US 2021/0130464, both incorporated herein by reference in their entireties). This was a dose response study with nine arms including a negative (no AAV9) control, then AAV9 in conjunction with placebo, PRV-3279 alone at 3 dose levels, co-administration of sirolimus with the three dose levels of PRV-3279, and sirolimus alone (Table 1). Longitudinal sampling included levels of IgM, IgG, AAV9 vector copy and tissue transgene activity at the end of study.

### Specific Study Objectives:

1. Amount of AAV9 vector detectable in blood (rate of clearance of vector)
2. Amount of GAA transgene detectable in tissue (efficiency of gene therapy transfection)
3. Levels of anti-AAV9 antibody in blood (immunogenicity of AAV vector)
4. Total IgM (PD marker of PRV-3279 activity)

TABLE 1
Design of PRV-3279 Dose Response Study

| Group | Mouse Strain | AAV9-DES-GAA | Rx Regimen for reduction in |
|---|---|---|---|
| #: | (n = 6-7) | Day 1 Dose | immunogenicity (mg/kg) |
| 1 | huCD32B | None | None |
| 2 | huCD32B | 6 × 10¹³ μg/Kg | No Rx |
| 3 | huCD32B | 6 × 10¹³ μg/Kg | Sirolimus |
| 4 | huCD32B | 6 × 10¹³ μg/Kg | PRV-3279 (10) |
| 5 | huCD32B | 6 × 10¹³ μg/Kg | PRV-3279 (10) + Sirolimus |
| 6 | huCD32B | 6 × 10¹³ μg/Kg | PRV-3279 (20) |
| 7 | huCD32B | 6 × 10¹³ μg/Kg | PRV-3279 (20) + Sirolimus |
| 8 | huCD32B | 6 × 10¹³ μg/Kg | PRV-3279 (50) |
| 9 | huCD32B | 6 × 10¹³ μg/Kg | PRV-3279 (50) + Sirolimus |

The dose dependency and sustained B cell binding by PRV-3279 lead to durable inhibition of immunoglobulin production without depletion of any circulating immune cell subset, including B cells. Immunoglobulins reduced in peripheral blood include IgM, IgA, IgG and IgE. The inhibition can be observed in the absence or presence of antigen stimulation (e.g., vaccination). This is an advantageous safety feature of PRV-3279 as a non-depleting agent, so that the patient can retain circulating immune cells, such as B cells, as part of the functioning immune system. In contrast, patients receiving cell depleting agents (e.g., rituximab, ocrelizumab, inebilizumab) take a considerable time to recover the depleted cell types (e.g., up to a year). This is of particular importance in consideration of therapy for BTHS where immunodeficiency is present and the risk of severe infection remains throughout the lifespan. Immunocompetence is also of clinical significance for the primary vaccination series in children as BTHS is present from birth.

In another aspect, pharmaceutical compositions are provided that can be used in the methods disclosed herein, i.e., pharmaceutical compositions for reducing or suppressing immunogenicity in a subject in need thereof, e.g., while or after receiving BTHS gene therapy that causes significant immunogenicity, or because the subject had pre-existing immunogenicity to the biotherapeutic (e.g., in the case of pre-existing anti-AAV antibodies due to prior wild-type adenoviral infections, or due to prior exposure to rAAV therapy). In some embodiments, the compositions disclosed herein can be administered to a patient before receiving BTHS gene therapy so as to prevent immunogenicity and/or reduce pre-existing antibodies.

In some embodiments, the pharmaceutical composition comprises a B cell inhibitor as disclosed herein and a pharmaceutically acceptable carrier. The B cell inhibitor can be formulated with the pharmaceutically acceptable carrier into a pharmaceutical composition. Additionally, the pharmaceutical composition can include, for example, instructions for use of the composition for the treatment of patients to reduce or suppress immunogenicity in a subject in need thereof, e.g., while or after receiving a biologic agent that causes significant immunogenicity.

As used herein, “pharmaceutically acceptable carrier” includes any and all solvents, dispersion media, coatings, antibacterial and antifungal agents, isotonic and absorption delaying agents, buffers, and other excipients that are physiologically compatible. Preferably, the carrier is suitable for parenteral, oral, or topical administration. Depending on the route of administration, the active compound, e.g., small molecule or biologic agent, may be coated in a material to protect the compound from the action of acids and other natural conditions that may inactivate the compound.

Pharmaceutically acceptable carriers include sterile aqueous solutions or dispersions and sterile powders for the extemporaneous preparation of sterile injectable solutions or dispersion, as well as conventional excipients for the preparation of tablets, pills, capsules and the like. The use of such media and agents for the formulation of pharmaceutically active substances is known in the art. Except insofar as any conventional media or agent is incompatible with the active compound, use thereof in the pharmaceutical compositions provided herein is contemplated. Supplementary active compounds can also be incorporated into the compositions.

A pharmaceutically acceptable carrier can include a pharmaceutically acceptable antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers which may be employed in the pharmaceutical compositions provided herein include water, ethanol, polyols (such as glycerol, propylene glycol, polyethylene glycol, and the like), and suitable mixtures thereof, and injectable organic esters, such as ethyl oleate. When required, proper fluidity can be maintained, for example, by the use of coating materials, such as lecithin, by the maintenance of the required particle size in the case of dispersions, and by the use of surfactants. In many cases, it may be useful to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

These compositions may also contain functional excipients such as preservatives, wetting agents, emulsifying agents and dispersing agents.

Therapeutic compositions typically must be sterile, non-phylogenic, and stable under the conditions of manufacture and storage. The composition can be formulated as a solution, microemulsion, liposome, or other ordered structure suitable to high drug concentration.

Sterile injectable solutions can be prepared by incorporating the active compound in the required amount in an appropriate solvent with one or a combination of ingredients enumerated above, as required, followed by sterilization, e.g., by microfiltration. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation include vacuum drying and freeze-drying (lyophilization) that yield a powder of the active ingredient plus any additional desired ingredient from a previously sterile-filtered solution thereof. The active agent(s) may be mixed under sterile conditions with additional pharmaceutically acceptable carrier(s), and with any preservatives, buffers, or propellants which may be required.

Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation.

Exemplary dosage ranges for administration of an antibody include: 10-1000 mg (antibody)/kg (body weight of the patient), 10-800 mg/kg, 10-600 mg/kg, 10-400 mg/kg, 10-200 mg/kg, 30-1000 mg/kg, 30-800 mg/kg, 30-600 mg/kg, 30-400 mg/kg, 30-200 mg/kg, 50-1000 mg/kg, 50-800 mg/kg, 50-600 mg/kg, 50-400 mg/kg, 50-200 mg/kg, 100-1000 mg/kg, 100-900 mg/kg, 100-800 mg/kg, 100-700 mg/kg, 100-600 mg/kg, 100-500 mg/kg, 100-400 mg/kg, 100-300 mg/kg, and 100-200 mg/kg. Exemplary dosage schedules include once every three days, once every five days, once every seven days (i.e., once a week), once every 10 days, once every 14 days (i.e., once every two weeks), once every 21 days (i.e., once every three weeks), once every 28 days (i.e., once every four weeks), once a month, once every 5 weeks, and once every 6 weeks.

In some embodiments, an about 5-40 mg/kg, about 5-20 mg/kg or about 10 mg/kg per dose of PRV-3279 can be administered once every 2 weeks, once every 3 weeks, once every 4 weeks, once every 5 weeks or once every 6 weeks. One or more doses can be administered, such as 1 dose, 2 doses or 3 doses. Administration can be via IV infusion. Any combination of the foregoing (e.g., 3 doses of 10 mg/kg per dose, once every 4 weeks) can be used for the reduction of the immunogenicity of biotherapeutics including gene therapy products. In some embodiments, the first dose can be given 2-6 weeks (e.g., 4 weeks) before gene therapy, the second dose at around the same time of the gene therapy, and the third dose 2-6 weeks (e.g., 4 weeks) after gene therapy. Thereafter, the patient can be monitored by examining the amount of specific antibodies against gene therapy vector (e.g., rAAV) and/or the transgene. If no or little antibody can be detected, then there will be no need for additional PRV-3279. If significant amount of antibody is present, then one or more dose of PRV-3279 can be administered to further modulate immunogenicity.

It may be advantageous to formulate parenteral compositions in unit dosage form for ease of administration and uniformity of dosage. Unit dosage form as used herein refers to physically discrete units suited as unitary dosages for the patients to be treated; each unit contains a predetermined quantity of active agent calculated to produce the desired therapeutic effect in association with any required pharmaceutical carrier. The specification for unit dosage forms are dictated by and directly dependent on (a) the unique characteristics of the active compound and the particular therapeutic effect to be achieved, and (b) the limitations inherent in the art of compounding such as an active compound for the treatment of sensitivity in individuals.

Actual dosage levels of the active ingredients in the pharmaceutical compositions disclosed herein may be varied so as to obtain an amount of the active ingredient which is effective to achieve the desired therapeutic response for a particular patient, composition, and mode of administration, without being toxic to the patient. “Parenteral” as used herein in the context of administration means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion.

The phrases “parenteral administration” and “administered parenterally” as used herein refer to modes of administration other than enteral (i.e., via the digestive tract) and topical administration, usually by injection or infusion, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion. Intravenous injection and infusion are often (but not exclusively) used for antibody administration.

When agents provided herein are administered as pharmaceuticals, to humans or animals, they can be given alone or as a pharmaceutical composition containing, for example, 0.001 to 90% (e.g., 0.005 to 70%, e.g., 0.01 to 30%) of active ingredient in combination with a pharmaceutically acceptable carrier.

### Therapeutic Uses and Methods

The compositions disclosed herein can be used to reduce or suppress immunogenicity caused by BHTS gene therapy delivered by various means (e.g., AAV and other wild-type and recombinant vectors, lentivirus modified human stem cells). In some embodiments, the B cell immunomodulators disclosed herein can be used to improve rAAV (recombinant adeno associated virus) vector based viral delivery of transgene, e.g., for inherited enzyme deficiencies such as TAFAZZIN deficiency associated with BHTS.

In some embodiments, the B cell immunomodulators disclosed herein can be used to modulate a limiting immune response elicited by multiple routes of delivery (even in sites of perceived immune privilege), such as systemic, intra-muscular, ocular (requiring high local dose results in local immune response), and central nervous system (CNS) (where leakage of viral capsid from CNS induces a systemic response that diminishes AAV uptake in CNS).

In some embodiments, the B cell immunomodulators disclosed herein can be used to modulate multiple limiting immunological pathways that are B cell dependent, including:
Development of neutralizing antibodies (nAb)
Antibody dependent cell-mediated cytotoxicity
Antibody dependent Complement mediated cytotoxicity
Autonomous B-cell Activation, e.g., via Toll-like receptors (TLR)
B cell mediated co-stimulation of T cells
B cell mediated antigen presentation

In some embodiments, the B cell immunomodulators disclosed herein can be used to improve multiple AAV clinical applications through B cell modulation, such as repeat dosing and/or increased AAV dose.

In some embodiments, after administration of PRV-3279, the peak plasma concentrations occurred at the end of infusion of the bispecific molecule, and there was minimal accumulation upon multiple dosing. This shows that PRV-3279 has good pharmacokinetics properties.

In some embodiments, administration of the PRV-3279 bispecific agent can result in inhibition of its own immunogenicity, i.e., lower prevalence and/or titers of anti-drug antibodies (ADA) with increased doses of the drug. This is in contrast to other immune-modulators. In addition, this suggests that increased dose of PRV-3279 such as 20 mg/kg, 30 mg/kg or 40 mg/kg can be well tolerated without added immunogenicity.

In some embodiments, it has been observed that PRV-3279 ADA does not affect pharmacokinetics (PK), pharmacodynamics (PD), safety or efficacy. This is surprising because ADA usually affects at least PK and PD. Without being bound by theory, it has been hypothesized that ADA does not neutralize PRV-3279.

In some embodiments, the PRV-3279 bispecific agent, in a dose-dependent fashion, binds to most (e.g., >80-90%) B cells, including both naive and memory phenotypes, upon administration, and remains bound to at least 50% of the B cells for at least 4 weeks after last administration of certain higher dosages of the drug. This shows sustained durability of the PD effect of PRV-3279, and supports once every month (or longer) administration.

In some embodiments, the dose dependency and sustained B cell binding by the PRV-3279 bispecific drug leads to durable inhibition of immunoglobulin production in the absence of depletion of any circulating cell subset, including B cells. Immunoglobulins reduced in peripheral blood include IgM, IgA, IgG and IgE. The inhibition can be observed in the absence or presence (e.g., vaccination) of antigen stimulation. This is an advantageous safety feature of PRV-3279 as a non-depleting agent, so that the patient can retain the circulating cells such as B cells to function as part of the immune system. In contrast, patients receiving depleting agents (e.g., rituximab, ocrelizumab, inebilizumab) take a long time to recover (e.g., a year).

In some embodiments, the BHTS gene therapy can be provided via AAV-Des-TAZ, a recombinant AAV vector serotype 9 (AAV9) which contains a full-length, codon-optimized, human TAFAZZIN gene. The serotype chosen is less prevalent in the human virome lowering the chance of individuals have pre-treatment antibodies circulating from previous viral infections. Pre-clinical trials were performed verifying clinical efficacy in knockdown (KD) mouse model of BTHS (Suzuki-Hatano 2019a). In mice, the vector penetrates tissues of the recipients and leads to transgene expression and production of TAFFAZIN gene product in skeletal and cardiac myocytes leading to a reversal of symptomatology.

Studies with the gene vectors in the KD BTHS mouse model demonstrated that changes in promoter led to variation in transcription and therefore tissue expression based on age in which injection was performed. This is an important concept when thinking about application in humans and optimal timing of administration or therapies that would allow for re-dosing at later time points.

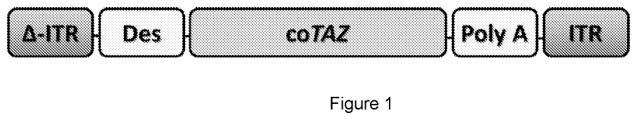

FIG. 1 is a schematic of an AAV-Des-TAZ vector design. The desmin promoter drives the expression of the full-length, human codon optimized TAZ cDNA+polyA tail. The sequence is flanked by AAV inverted terminal repeats (ITRs) with the left flanking ITR containing the necessary alterations to yield dsAAV.

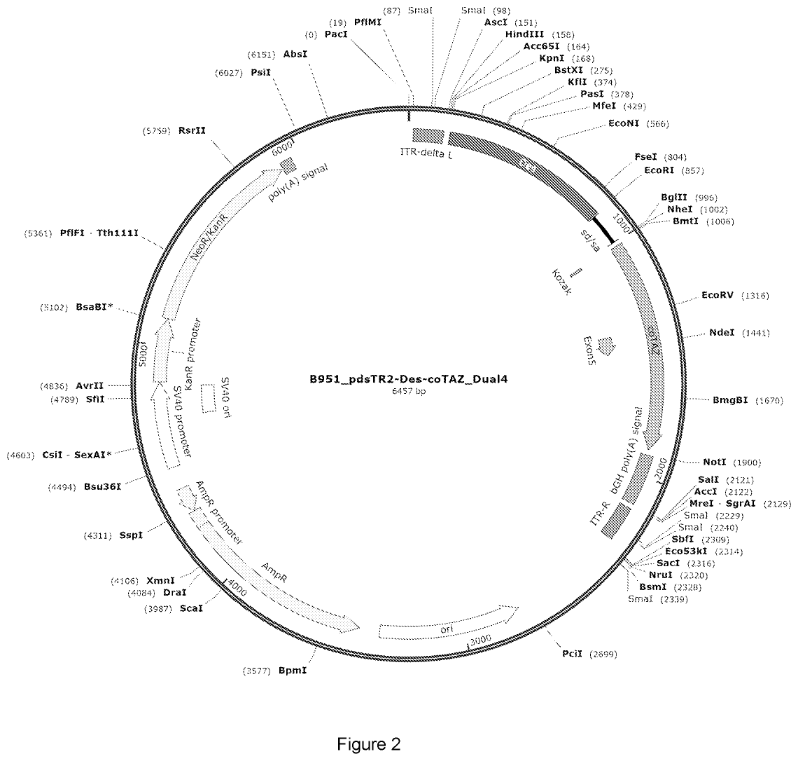

FIG. 2 is an exemplary construct B951 (pds2TR-Des-coTAZ_Dual4). B951 is the self-complementary construct for codon optimized full length tafazin controlled by the Desmin promoter. Sequence is shown in Table 2.

TABLE 2
B951 (pds2TR-Des-coTAZ_Dua14)

| Elements (5′ → | | AA sequence (as |
|---|---|---|
| 3′) | NT sequence | applicable) |
| ITR-delta L | TTGGCCACTCCCTCTCTGCGCGCTC | |
| | GCTCGCTCACTGAGGCCGGGCGAC | |
| | CAAAGGTCGCCCGACGCCCGGGCT | |
| | TTGCCCGGGCGGCCTCAGTGAGCG | |
| | AGCGAGCGCGCAGAGAGGGAGTGG | |
| | CCA (SEQ ID NO: 8) | |
| Des promoter | CCCTGCCCCCACAGCTCCTCTCCTG | |
| | TGCCTTGTTTCCCAGCCATGCGTTCT | |
| | CCTCTATAAATACCCGCTCTGGTATT | |
| | TGGGGTTGGCAGCTGTTGCTGCCAG | |
| | GGAGATGGTTGGGTTGACATGCGGC | |
| | TCCTGACAAAACACAAACCCCTGGT | |
| | GTGTGTGGGCGTGGGTGGTGTGAG | |
| | TAGGGGGATGAATCAGGGAGGGGG | |
| | CGGGGGACCCAGGGGGCAGGAGCC | |
| | ACACAAAGTCTGTGCGGGGGTGGG | |
| | AGCGCACATAGCAATTGGAAACTGA | |
| | AAGCTAATCAGACCCTTTCTGGAAAT | |
| | CAGCCCACTGTTTATATACTTGAGGC | |
| | CCCACCCTCGAGATAACCAGGGCTG | |
| | AAAGAGGCCCGCCTGGGGGCTGCA | |
| | GACATGCTTGCTGCCTGCCCTGGCG | |
| | AAGGATTGGCAGGCTTGCCCGTCAC | |
| | AGGACCCCCGCTGGCTGACTCAGG | |
| | GGCGCAGGCCTCTTGCGGGGGAGC | |
| | TGGCCTCCCCGCCCCCACGGCCAC | |
| | GGGCCGCCCTTTCCTGGCAGGACA | |
| | GCGGGATCTTGCAGCTGTCAGGGG | |
| | AGGGGAGGCGGGGGCTGATGTCAG | |
| | GAGGGATACAAATAGTGCCGACGGC | |
| | TGGGGGCCCTGTCTCCCCTCGCCG | |
| | CATCCACTCTCCGGCCGGCCGCCTG | |
| | CCCGCCGCCTCCTCCGTGCGCCCG | |
| | CCAGCCTCGCCCGCGCCGTCA (SEQ | |
| | ID NO: 9) | |
| chimeric intron | GTATCAAGGTTACAAGACAGGTTTAA | |
| | GGAGACCAATAGAAACTGGGCTTGT | |
| | CGAGACAGAGAAGACTCTTGCGTTT | |
| | CTGATAGGCACCTATTGGTCTTACTG | |
| | ACATCCACTTTGCCTTTCTCTCCACA | |
| | GGCC (SEQ ID NO: 10) | |
| COTAZ | ATGCCCCTGCACGTGAAGTGGCCTT | MPLHVKWPFPAVPPL |
| | TCCCTGCCGTGCCTCCTCTGACCTG | TWTLASSVVMGLVGT |
| | GACACTGGCTTCCAGCGTCGTGATG | YSCFWTKYMNHLTV |
| | GGCCTCGTGGGCACCTACAGCTGCT | HNREVLYELIEKRGP |
| | TTTGGACCAAGTACATGAACCACCT | ATPLITVSNHQSCMD |
| | GACCGTGCACAACAGAGAGGTGCTG | DPHLWGILKLRHIWN |
| | TACGAGCTGATCGAGAAGAGAGGCC | LKLMRWTPAAADICF |
| | CTGCCACCCCCCTGATCACCGTGTC | TKELHSHFFSLGKCV |
| | CAATCACCAGAGCTGCATGGACGAC | PVCRGAEFFQAENE |
| | CCCCACCTGTGGGGCATCCTGAAGC | GKGVLDTGRHMPGA |
| | TGCGGCACATCTGGAATCTGAAGCT | GKRREKGDGVYQKG |
| | GATGCGGTGGACCCCCGCTGCCGC | MDFILEKLNHGDWVH |
| | CGATATCTGCTTCACCAAAGAGCTG | IFPEGKVNMSSEFLR |
| | CACAGCCACTTTTTCAGCCTGGGGA | FKWGIGRLIAECHLN |
| | AGTGCGTGCCCGTGTGCAGAGGCG | PIILPLWHVGMNDVLP |
| | CCGAGTTCTTCCAGGCCGAGAATGA | NSPPYFPRFGQKITV |
| | GGGAAAGGGCGTGCTGGACACCGG | LIGKPFSALPVLERLR |
| | CAGACATATGCCTGGCGCCGGAAAG | AENKSAVEMRKALTD |
| | CGCAGAGAAAAGGGCGACGGCGTG | FIQEEFQHLKTQAEQ |
| | TACCAGAAAGGCATGGACTTCATCC | LHNHLQPGR (SEQ ID |
| | TGGAAAAGCTGAACCACGGCGACTG | NO: 12) |
| | GGTGCACATCTTCCCCGAGGGCAAA | |
| | GTGAATATGAGCAGCGAGTTCCTGC | |
| | GGTTCAAATGGGGCATCGGCCGGCT | |
| | GATCGCCGAGTGCCACCTGAACCCT | |
| | ATCATCCTGCCCCTGTGGCACGTGG | |
| | GCATGAACGACGTGCTGCCCAACAG | |
| | CCCCCCCTACTTCCCTAGATTCGGC | |
| | CAGAAAATCACCGTGCTGATCGGCA | |
| | AGCCCTTCAGCGCCCTGCCCGTGCT | |
| | GGAAAGACTGCGGGCCGAGAACAA | |
| | GAGCGCCGTGGAAATGAGAAAGGC | |
| | CCTGACCGATTTCATCCAGGAAGAG | |
| | TTCCAGCACCTGAAAACACAGGCCG | |
| | AGCAGCTGCACAACCATCTGCAGCC | |
| | CGGCAGATGA (SEQ ID NO: 11) | |
| bGH polyA | CTGTGCCTTCTAGTTGCCAGCCATC | |
| | TGTTGTTTGCCCCTCCCCCGTGCCT | |
| | TCCTTGACCCTGGAAGGTGCCACTC | |
| | CCACTGTCCTTTCCTAATAAAATGAG | |
| | GAAATTGCATCGCATTGTCTGAGTA | |
| | GGTGTCATTCTATTCTGGGGGGTGG | |
| | GGTGGGGCAGGACAGCAAGGGGGA | |
| | GGATTGGGAAGACAATAGCAGGCAT | |
| | GCTGGGGA (SEQ ID NO: 13) | |
| ITR-R | AGGAACCCCTAGTGATGGAGTTGGC | |
| | CACTCCCTCTCTGCGCGCTCGCTCG | |
| | CTCACTGAGGCCGGGCGACCAAAG | |
| | GTCGCCCGACGCCCGGGCTTTGCC | |
| | CGGGCGGCCTCAGTGAGCGAGCGA | |
| | GCGCGCAGAGAGGGAGTGGCCAA | |
| | (SEQ ID NO: 14) | |

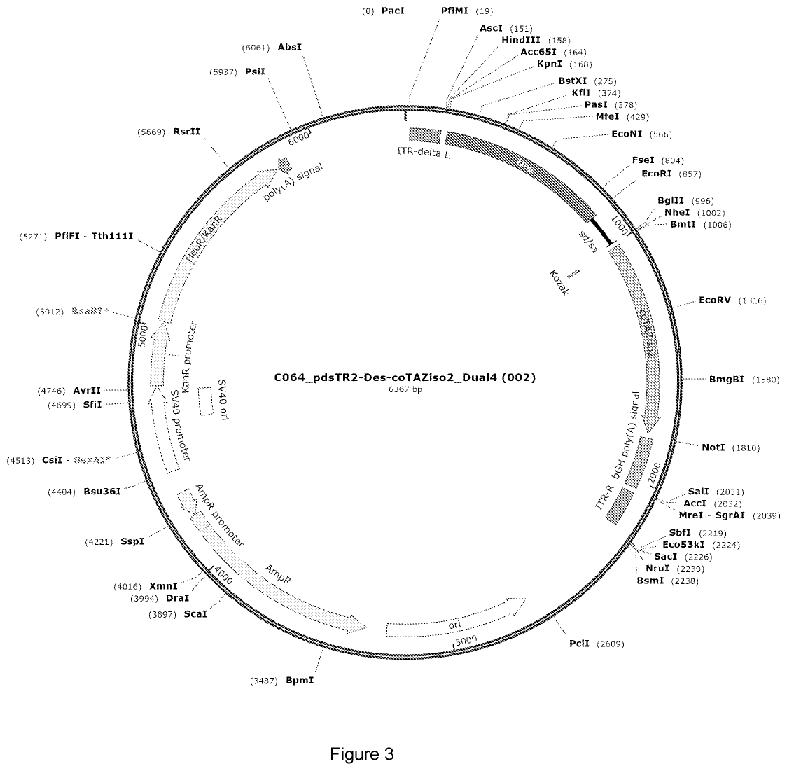

FIG. 3 is an exemplary construct C064 (pds2TR-Des-coTAZiso2_Dual4). C064 substitutes the delta5 or exon 5 deleted tafazin cDNA. Sequence is shown in Table 3.

TABLE XX
C064 (pds2TR-Des-coTAZiso2_Dua14)

| Elements (5' → | | AA sequence (as |
|---|---|---|
| 3') | NT sequence | applicable) |
| ITR-delta L | TTGGCCACTCCCTCTCTGCGCGCTC | |
| | GCTCGCTCACTGAGGCCGGGCGAC | |
| | CAAAGGTCGCCCGACGCCCGGGCT | |
| | TTGCCCGGGCGGCCTCAGTGAGCG | |
| | AGCGAGCGCGCAGAGAGGGAGTGG | |
| | CCA (SEQ ID NO: 8) | |
| Des promoter | CCCTGCCCCCACAGCTCCTCTCCTG | |
| | TGCCTTGTTTCCCAGCCATGCGTTCT | |
| | CCTCTATAAATACCCGCTCTGGTATT | |
| | TGGGGTTGGCAGCTGTTGCTGCCAG | |
| | GGAGATGGTTGGGTTGACATGCGGC | |
| | TCCTGACAAAACACAAACCCCTGGT | |
| | GTGTGTGGGCGTGGGTGGTGTGAG | |
| | TAGGGGGATGAATCAGGGAGGGGG | |
| | CGGGGGACCCAGGGGGCAGGAGCC | |
| | ACACAAAGTCTGTGCGGGGGTGGG | |
| | AGCGCACATAGCAATTGGAAACTGA | |
| | AAGCTAATCAGACCCTTTCTGGAAAT | |
| | CAGCCCACTGTTTATATACTTGAGGC | |
| | CCCACCCTCGAGATAACCAGGGCTG | |
| | AAAGAGGCCCGCCTGGGGGCTGCA | |
| | GACATGCTTGCTGCCTGCCCTGGCG | |
| | AAGGATTGGCAGGCTTGCCCGTCAC | |
| | AGGACCCCCGCTGGCTGACTCAGG | |
| | GGCGCAGGCCTCTTGCGGGGGAGC | |
| | TGGCCTCCCCGCCCCCACGGCCAC | |
| | GGGCCGCCCTTTCCTGGCAGGACA | |
| | GCGGGATCTTGCAGCTGTCAGGGG | |
| | AGGGGAGGCGGGGGCTGATGTCAG | |
| | GAGGGATACAAATAGTGCCGACGGC | |
| | TGGGGGCCCTGTCTCCCCTCGCCG | |
| | CATCCACTCTCCGGCCGGCCGCCTG | |
| | CCCGCCGCCTCCTCCGTGCGCCCG | |
| | CCAGCCTCGCCCGCGCCGTCA (SEQ | |
| | ID NO: 9) | |
| chimeric intron | GTATCAAGGTTACAAGACAGGTTTAA | |
| | GGAGACCAATAGAAACTGGGCTTGT | |
| | CGAGACAGAGAAGACTCTTGCGTTT | |
| | CTGATAGGCACCTATTGGTCTTACTG | |
| | ACATCCACTTTGCCTTTCTCTCCACA | |
| | GGCC (SEQ ID NO: 10) | |
| coTAZiso2 | ATGCCCCTGCACGTGAAGTGGCCTT | MPLHVKWPFPAVPPL |
| | TCCCTGCCGTGCCTCCTCTGACCTG | TWTLASSVVMGLVGT |
| | GACACTGGCTTCCAGCGTCGTGATG | YSCFWTKYMNHLTV |
| | GGCCTCGTGGGCACCTACAGCTGCT | HNREVLYELIEKRGP |
| | TTTGGACCAAGTACATGAACCACCT | ATPLITVSNHQSCMD |
| | GACCGTGCACAACAGAGAGGTGCTG | DPHLWGILKLRHIWN |
| | TACGAGCTGATCGAGAAGAGAGGCC | LKLMRWTPAAADICF |
| | CTGCCACCCCCCTGATCACCGTGTC | TKELHSHFFSLGKCV |
| | CAATCACCAGAGCTGCATGGACGAC | PVCRGDGVYQKGMD |
| | CCCCACCTGTGGGGCATCCTGAAGC | FILEKLNHGDWVHIFP |
| | TGCGGCACATCTGGAATCTGAAGCT | EGKVNMSSEFLRFK |
| | GATGCGGTGGACCCCCGCTGCCGC | WGIGRLIAECHLNPIIL |
| | CGATATCTGCTTCACCAAAGAGCTG | PLWHVGMNDVLPNS |
| | CACAGCCACTTTTTCAGCCTGGGGA | PPYFPRFGQKITVLIG |
| | AGTGCGTGCCCGTGTGCAGAGGCG | KPFSALPVLERLRAE |
| | ACGGCGTGTACCAGAAAGGCATGGA | NKSAVEMRKALTDFI |
| | CTTCATCCTGGAAAAGCTGAACCAC | QEEFQHLKTQAEQLH |
| | GGCGACTGGGTGCACATCTTCCCCG | NHLQPGR (SEQ ID |
| | AGGGCAAAGTGAATATGAGCAGCGA | NO: 16) |
| | GTTCCTGCGGTTCAAATGGGGCATC | |
| | GGCCGGCTGATCGCCGAGTGCCAC | |
| | CTGAACCCTATCATCCTGCCCCTGT | |
| | GGCACGTGGGCATGAACGACGTGC | |
| | TGCCCAACAGCCCCCCCTACTTCCC | |
| | TAGATTCGGCCAGAAAATCACCGTG | |
| | CTGATCGGCAAGCCCTTCAGCGCCC | |
| | TGCCCGTGCTGGAAAGACTGCGGG | |
| | CCGAGAACAAGAGCGCCGTGGAAAT | |
| | GAGAAAGGCCCTGACCGATTTCATC | |
| | CAGGAAGAGTTCCAGCACCTGAAAA | |
| | CACAGGCCGAGCAGCTGCACAACCA | |
| | TCTGCAGCCCGGCAGATGA (SEQ ID | |
| | NO: 15) | |
| bGH polyA | CTGTGCCTTCTAGTTGCCAGCCATC | |
| | TGTTGTTTGCCCCTCCCCCGTGCCT | |
| | TCCTTGACCCTGGAAGGTGCCACTC | |
| | CCACTGTCCTTTCCTAATAAAATGAG | |
| | GAAATTGCATCGCATTGTCTGAGTA | |
| | GGTGTCATTCTATTCTGGGGGGTGG | |
| | GGTGGGGCAGGACAGCAAGGGGGA | |
| | GGATTGGGAAGACAATAGCAGGCAT | |
| | GCTGGGGA (SEQ ID NO: 13) | |
| ITR-R | AGGAACCCCTAGTGATGGAGTTGGC | |
| | CACTCCCTCTCTGCGCGCTCGCTCG | |
| | CTCACTGAGGCCGGGCGACCAAAG | |
| | GTCGCCCGACGCCCGGGCTTTGCC | |
| | CGGGCGGCCTCAGTGAGCGAGCGA | |
| | GCGCGCAGAGAGGGAGTGGCCAA | |
| | (SEQ ID NO: 14) | |

In some embodiments, a combination therapy of PRV-3279 with AAV-Des-TAZ gene therapy is provided. As disclosed herein, the effect of PRV-3279 on B-cell function can be transient. This allows for the gene therapy to be delivered and for immunogenicity to remain low while not depleting B-cell populations and therefore not increasing risk of infection long term. PRV-3279 in an ideal candidate for use in combination with gene therapy by reducing immunogenicity, and thereby enhancing immediate effectiveness of gene vector while also potentially allowing for re-dosing of the gene therapy at a later timepoint. The reduced formation of anti-viral vector antibodies is particularly important for pediatric patients who will continue to have expected tissue and organ growth necessitating repeat delivery of gene therapy. In addition, there is data which support that PRV-3279 may help inhibit naïve B-cells thus reducing the immune response with initial exposure to the gene vector.

Based on knowledge of mechanism for PRV-3279 and understanding of immunogenicity of gene therapy, one exemplary plan can include dosing PRV-3279 three times during the course of a treatment of AAV-Des-TAZ as follows: 1 week prior to vector delivery, 2 weeks after delivery, and 3-4 weeks following 2^{nd} dose.

### EXAMPLES

The following examples, including the experiments conducted and results achieved, are provided for illustrative purposes only and are not to be construed as limiting the disclosure.

### Example 1: Clinical Trial Design with Adults

Provided herein is a 96 week, 4×4, double crossover Phase I/II clinical trial to test the safety and effectiveness of two systemic doses—3×10¹³ μg/kg and 1×10¹⁴ μg/kg—of gene therapy (AAV-Des-TAZ) with standard prophylaxis and with Sirolimus+PRV-3279 dosed at −1 week, +2 weeks, +4 weeks in 4 (n=8 total) cohorts of adolescents and young adults with BTHS (ages 15-40 years).

Subjects with standard prophylaxis can receive rituximab (Genentech, dose 375 mg/m2) weekly starting at −21 days prior to dosing. Pre-medication will be administered 30-60 minutes prior to rituximab and can include acetaminophen (Tylenol) and diphenhydramine (Benadryl). In addition, subjects can receive sirolimus (Wyeth, dose 0.6-1 mg/m2/day, adjusted to maintain a trough serum sirolimus level of 3-7 ng/mL) everyday starting at −7 days prior to dosing until Week 12 post-dose.

Subjects with experimental prophylaxis can receive PRV-3279 starting at −7 days prior to dosing, +14 days, and +28 days. Pre-medication can be administered 30-60 minutes prior to PRV-3279 and can include acetaminophen (Tylenol) and diphenhydramine (Benadryl). In addition, subjects can receive sirolimus (Wyeth, dose 0.6-1 mg/m2/day, adjusted to maintain a trough serum sirolimus level of 3-7 ng/mL) everyday starting at −7 days prior to dosing until Week 12 post-dose.

Subjects can be monitored for infection through physical exams and periodic laboratory assessments and can be treated appropriately if deemed clinically necessary.

The primary safety objective of this study is to investigate the safety and tolerability of systemic IV delivery of rAAV9-Des-TAZ with PRV-3279 in BTHS. Safety outcomes can include immune system responses, heart function at rest, heart arrhythmias and major adverse cardiac events.

Clinical objectives specific to AAV-Des-TAZ include: (1) To test the safety and tolerability of systemic delivery of AAV-Des-TAZ, and (2) To test the efficacy (VO2peak) of systemic delivery of AAV-Des-TAZ.

Clinical objectives specific to PRV-3279 include: (1) Levels of anti-AAV9 antibody in blood (immunogenicity of AAV vector), and (2) Total IgM (PD marker of PRV-3279 activity).

In Cohort A (n=4), 3 subjects can be randomized to receive a one-time, single low dose of 3×10¹³ μg/kg of AAV-Des-TAZ and one subject can be randomized to receive a one-time, single high dose of 1×10¹⁴ μg/kg. This can be performed with standard prophylaxis to anticipate re-dosing with (rituximab and sirolimus.

After 16 weeks, the 3 subjects that received the low dose on Day 1 can crossover and receive the inverse or high dose on Week 16.

The one subject who received the high dose on Day 1 can receive placebo on Week 16.

In Cohort B (n=4), 3 subjects can be randomized to receive a one-time, single high dose of 1×10¹⁴ μg/kg AAV9-Des-TAZ and one subject to one-time, single low dose of 3×10¹³ μg/kg of rAAV9-Des-TAZ. After 16 weeks, the one subject that received the low dose on Week 16 can crossover and receive the high dose on Week 32. The 3 subjects who received the high dose on Week 16 can receive placebo on Week 32. This can be performed with standard prophylaxis to anticipate re-dosing with rituximab and sirolimus.

In Cohort C (n=4), 3 subjects can be randomized to receive a one-time, single low dose of 3×10¹³ μg/kg of AAV-Des-TAZ and one subject can be randomized to receive a one-time, single high dose of 1×10¹⁴ μg/kg. This can be performed with experimental prophylaxis to anticipate re-dosing with PRV-3279 and sirolimus.

In Cohort D (n=4), 3 subjects can be randomized to receive a one-time, single high dose of 1×10¹⁴ μg/kg AAV9-Des-TAZ and one subject to one-time, single low dose of 3×10¹³ μg/kg of rAAV9-Des-TAZ. After 16 weeks, the one subject that received the low dose on Week 16 can crossover and receive the high dose on Week 32. The 3 subjects who received the high dose on Week 16 can receive placebo on Week 32. This can be performed with experimental prophylaxis to anticipate re-dosing with PRV-3279 and sirolimus.

### Example 2: Toxicology Study Design for Combination Therapy of PRV-3279 with AAV-Des-TAZ Gene Therapy

120 age-matched huCD32B mice can be used in this study.

PRV-3279 surrogate requirements:

$$6 groups \times 28 days \times 10 mice \times 25 g mouse \times 50 mg / kg = 2100 mg$$

$$6 groups \times 84 days \times 10 mice \times 25 g mouse \times 50 mg / kg = 6300 mg$$

$$+ 30 \% overage = \sim 11 g of PRV - 3279 surrogate .$$

Toxicology study can be performed in mice having received low dose (e.g., 3×10¹³ μg/kg) or high dose (e.g., 1×10¹⁴ μg/kg) of gene therapy (AAV-Des-TAZ) 50 mg/kg/day PRV-3279 for 28 days or 84 days, in accordance with the schedule below.

| | | | Subgroup | | |
|---|---|---|---|---|---|
| | Dose | | (sacrificetime | | |
| | (μg/kg) | Dose (mg/kg) | post initialvector | | N |
| Group | Vector | PRV-3279 (i.p.) | dosing) | Route | (M/F) |
| 1 | Vehicle | Vehicle | 1a (28 days) | IV | 5/5 |
| | | | 1b (84 days) | IV | 5/5 |
| 2 | Low Dose | Vehicle | 2a (28 days) | IV | 5/5 |
| | | | 2b (84 days) | IV | 5/5 |
| 3 | High Dose | Vehicle | 4a (28 days) | IV | 5/5 |
| | | | 4b (84 days) | IV | 5/5 |
| 4 | Vehicle | 50 mg/kg/day | 1a (28 days) | IV | 5/5 |
| | | | 1b (84 days) | IV | 5/5 |
| 5 | Low Dose | 50 mg/kg/day | 2a (28 days) | IV | 5/5 |
| | | | 2b (84 days) | IV | 5/5 |
| 6 | High Dose | 50 mg/kg/day | 4a (28 days) | IV | 5/5 |
| | | | 4b (84 days) | IV | 5/5 |

### MODIFICATIONS

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

### REFERENCES

Barth, P G et al. “An X-linked mitochondrial disease affecting cardiac muscle, skeletal muscle and neutrophil leucocytes.” Journal of the neurological sciences vol. 62, 1-3 (1983): 32.7-55. doi:10.1016/0022-510x(83)90209-5
Barth Syndrome Foundation, 2018. The Voice of the Patient: Barth Syndrome [WWW Document]. www.fda.gov/media/130562/download
Corti, M. et al., 2014. B-Cell Depletion is Protective Against Anti-AAV Capsid Immune Response: A Human Subject Case Study. Mol. Ther. Methods Clin. Dev. 1. doi.org/10.1038/mtm.2014.33
Corti, Manuela et al., “Safety of Intradiaphragmatic Delivery of Adeno-Associated Virus-Mediated Alpha-Glucosidase (rAAV1-CMV-hGAA) Gene Therapy in Children Affected by Pompe Disease.” Human gene therapy. Clinical development vol. 28, 4 (2017): 208-218. doi:10.1089/humc.2017.146
Gonzalez I L, Human tafazzin (TAZ) gene mutation and variation database. 2012. Science and Research section of www.barthsyndrome.org
Rigaud, C., Lebre, A S., Touraine, R. et al. Natural history of Barth syndrome: a national cohort study of 22 patients. Orphanet J Rare Dis 8, 70 (2013). doi.org/10.1186/1750-1172-8-70
Roberts A E et al., 2012. The Barth Syndrome Registry: Distinguishing disease characteristics and growth data from a longitudinal study. Am J Med Genet Part A 158A:2726-2732.
Rugolotto, Simone et al. “Long-term treatment of Barth syndrome with pantothenic acid: a retrospective study.” Molecular genetics and metabolism vol. 80, 4 (2003): 408-11. doi:10.1016/j.ymgme.2003.07.002
Nathwani A. C. et al. (2007). Safe and efficient transduction of the liver after peripheral vein infusion of self-complementary AAV vector results in stable therapeutic expression of human FIX in nonhuman primates. Blood 109, 1414-1421
Steward, C. Barth Syndrome, Orphanet Review, June 2011.
McKusick, V., OMIM entry #302060, September 2018.

1. A method of reducing immunogenicity associated with gene therapy for Barth Syndrome (BTHS), comprising administering to a patient receiving or having received a BTHS gene therapy, an effective amount of B cell inhibitor that is non-depletional.

2. The method of claim 1, wherein the BTHS gene therapy comprises administering to the patient genetically modified cells having a recombinant adeno-associated virus (rAAV) vector encoding a TAFAZZIN transgene.

3. The method of claim 1, wherein the B cell inhibitor is a CD32B×CD79B bi-specific antibody capable of immunospecifically binding an epitope of CD32B and an epitope of CD79B.

4. The method of claim 3, wherein the CD32B×CD79B bi-specific antibody comprises:
(A) a VL_{CD32B} domain that comprises the amino acid sequence of SEQ ID NO: 1;
(B) a VH_{CD32B} domain that comprises the amino acid sequence of SEQ ID NO: 2;
(C) a VL_{CD79B} domain that comprises the amino acid sequence of SEQ ID NO: 3; and
(D) a VH_{CD79B} domain that comprises the amino acid sequence of SEQ ID NO: 4.

5. The method of claim 4, wherein said CD32B×CD79B bi-specific antibody is an Fc diabody comprising:
(A) a first polypeptide chain that comprises the amino acid sequence of SEQ ID NO: 5;
(B) a second polypeptide chain that comprises the amino acid sequence of SEQ ID NO: 6; and
(C) a third polypeptide chain that comprises the amino acid sequence of SEQ ID NO: 7.

6. The method of claim 5, comprising administering the Fc diabody at a dose of between about 5 mg/kg and about 100 mg/kg, or between about 5 mg/kg and about 50 mg/kg, or between about 5 mg/kg and about 40 mg/kg, and at a dosage regimen of between one dose per week and one dose per 6 weeks.

7. The method of claim 5, comprising administering the Fc diabody at a dose of about 10 mg/kg, and at a dosage regimen of one dose per 1-4 weeks.

8. The method of claim 5, comprising administering 3 doses of the Fc diabody at a dose of about 10 mg/kg, 1 week prior to a first BTHS gene therapy delivery, 2 weeks after the first BTHS gene therapy delivery, and 3-4 weeks following a second BTHS gene therapy delivery.

9. The method of claim 8, comprising administering a first dose about 2 days to about 6 weeks prior to administration of the BTHS gene therapy, a second dose at about the same time as administration of the BTHS gene therapy, and a third dose about 2 days to about 6 weeks after administration of the BTHS gene therapy.

10. The method of claim 5, wherein the Fc diabody results in inhibition of its own immunogenicity upon administration, with lower prevalence and/or titers of anti-drug antibodies (ADA) at increased doses.

11. The method of claim 10, wherein the ADA does not neutralize the Fc diabody.

12. The method of claim 5, wherein the Fc diabody, in a dose-dependent fashion, binds to at least 80% B cells upon administration, and remains bound to at least 50% of the B cells for at least 4 weeks after last administration.

13. The method of claim 5, wherein the Fc diabody results in sustained inhibition of immunoglobulin production without depleting circulating B cells.

14. The method of claim 13, wherein the immunoglobulin includes one or more of IgM, IgA, IgG and IgE.

15. The method of claim 1, further comprising monitoring the patient by examining the presence of specific antibodies against BTHS gene therapy.

16. The method of claim 15, further comprising administering one or more dose of the B cell inhibitor to further modulate immunogenicity.

17. The method of claim 5, further comprising co-administering one or more immune-modulators.

18. The method of claim 17, wherein the one or more immune-modulators are selected from sirolimus, rapamycin, abatacept, teplizumab and immunoglobulin G-degrading enzyme of Streptococcus pyogenes.

19. The method of claim 5, further comprising co-administering sirolimus.

20. Use of a B cell inhibitor that is non-depletional in the manufacture of a medicament for reducing immunogenicity associated with Barth Syndrome (BTHS) gene therapy, wherein optionally the BTHS gene therapy comprises genetically modified cells having a recombinant adeno-associated virus (rAAV) vector encoding a TAFAZZIN transgene.

21. A pharmaceutical composition for reducing immunogenicity associated with gene therapy for Barth Syndrome (BTHS), comprising an effective amount of a CD32B×CD79B bi-specific antibody capable of immunospecifically binding an epitope of CD32B and an epitope of CD79B, administered prior to, concurrently with, and/or after BTHS gene therapy, wherein optionally the BTHS gene therapy comprises genetically modified cells having a recombinant adeno-associated virus (rAAV) vector encoding a TAFAZZIN transgene.

22. The pharmaceutical composition of claim 21, wherein the CD32B×CD79B bi-specific antibody comprises:
(A) a VL_{CD32B} domain that comprises the amino acid sequence of SEQ ID NO: 1;
(B) a VH_{CD32B} domain that comprises the amino acid sequence of SEQ ID NO: 2;
(C) a VL_{CD79B} domain that comprises the amino acid sequence of SEQ ID NO: 3; and
(D) a VH_{CD79B} domain that comprises the amino acid sequence of SEQ ID NO: 4.

23. The pharmaceutical composition of claim 22, wherein said CD32B×CD79B bi-specific antibody is an Fc diabody comprising:
(A) a first polypeptide chain that comprises the amino acid sequence of SEQ ID NO: 5;
(B) a second polypeptide chain that comprises the amino acid sequence of SEQ ID NO: 6; and
(C) a third polypeptide chain that comprises the amino acid sequence of SEQ ID NO: 7.

24. The pharmaceutical composition of claim 23, comprising the Fc diabody at a dose of between about 5 mg/kg and about 100 mg/kg, or between about 5 mg/kg and about 50 mg/kg, or between about 5 mg/kg and about 40 mg/kg, and at a dosage regimen of between one dose per week and one dose per 6 weeks.

25. The pharmaceutical composition of claim 23, comprising the Fc diabody at a dose of about 10 mg/kg, at one dose per 1-4 weeks, preferably at a regimen of 1 week prior to a first BTHS gene therapy delivery, 2 weeks after the first BTHS gene therapy delivery, and 3-4 weeks following a second BTHS gene therapy delivery.

26. The pharmaceutical composition of claim 25, comprising a first dose about 2 days to about 6 weeks prior to administration of the BTHS gene therapy, a second dose at about the same time as administration of the BTHS gene therapy, and a third dose about 2 days to about 6 weeks after administration of the BTHS gene therapy.

27. The pharmaceutical composition of claim 21, further comprising one or more immune-modulators selected from sirolimus, rapamycin, abatacept, teplizumab and immunoglobulin G-degrading enzyme of Streptococcus pyogenes.


SEQUENCE LISTING (16 sequences)

SEQ ID NO: 1 - protein, 106 aa, synthetic construct
DIQMTQSPSSLSASGDRVTITCRASQEISGYLSWLQQKPGKAPRRLIYAASTLDSGVPSRFSGSESGTEFTLTISSLQPEDFATYYCLQYFSYPLTFGGGTKVEIK

SEQ ID NO: 2 - protein, 116 aa, synthetic construct
EVQLVESGGGLVQPGGSLRLSCAASGFTFSDAWMDWVRQAPGKGLEWVAEIRNKAKNHATYYAESVIGRFTISRDDAKNSLYLQMNSLRAEDTAVYYCGALGLDYWGQGTLVTVSS

SEQ ID NO: 3 - protein, 111 aa, synthetic construct
DVVMTQSPLSLPVTLGQPASISCKSSQSLLDSDGKTYLNWFQQRPGQSPNRLIYLVSKLDSGVPDRFSGSGSGTDFTLKISRVEAEDGVYYCWQGTHFPLTFGGGTKLEIK

SEQ ID NO: 4 - protein, 113 aa, synthetic construct
QVQLVQSGAEVKKPGASVKVSCKASGYTFTSYWMNWVRQAPGQGLEWIGMIDPSDSETHYNQKFKDRVTMTTDTSTSTAYMELRSLRSDDTAVYYCARAMGYWGQGTTVTVSS

SEQ ID NO: 5 - protein, 501 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAPSSSPMEDIQMTQSPSSLSASGDRVTITCRASQEISGYLSWLQQKPGKAPRRLIYAASTLDSGVPSRFSGSESGTEFTLTISSLQPEDFATYYCLQYFSYPLTFGGGTKVEIKGGGSGGGGQVQLVQSGAEVKKPGASVKVSCKASGYTFTSYWMNWVRQAPGQGLEWIGMIDPSDSETHYNQKFKDRVTMTTDTSTSTAYMELRSLRSDDTAVYYCARAMGYWGQGTTVTVSSGGCGGGEVAALEKEVAALEKEVAALEKEVAALEKGGGNS

SEQ ID NO: 6 - protein, 269 aa, synthetic construct
DVVMTQSPLSLPVTLGQPASISCKSSQSLLDSDGKTYLNWFQQRPGQSPNRLIYLVSKLDSGVPDRFSGSGSGTDFTLKISRVEAEDGVYYCWQGTHFPLTFGGGTKLEIKGGGSGGGGEVQLVESGGGLVQPGGSLRLSCAASGFTFSDAWMDWVRQAPGKGLEWVAEIRNKAKNHATYYAESVIGRFTISRDDAKNSLYLQMNSLRAEDTAVYYCGALGLDYWGQGTLVTVSSGGCGGGKVAALKEKVAALKEKVAALKEKVAALKE

SEQ ID NO: 7 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 8 - DNA, 124 bp, synthetic construct
  source 1..124: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggcca

SEQ ID NO: 9 - DNA, 687 bp, synthetic construct
  source 1..687: mol_type = other DNA
ccctgcccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagctaatcagaccctttctggaaatcagcccactgtttatatacttgaggccccaccctcgagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtca

SEQ ID NO: 10 - DNA, 132 bp, synthetic construct
  source 1..132: mol_type = other DNA
gtatcaaggttacaagacaggtttaaggagaccaatagaaactgggcttgtcgagacagagaagactcttgcgtttctgataggcacctattggtcttactgacatccactttgcctttctctccacaggcc

SEQ ID NO: 11 - DNA, 879 bp, synthetic construct
  source 1..879: mol_type = other DNA
atgcccctgcacgtgaagtggcctttccctgccgtgcctcctctgacctggacactggcttccagcgtcgtgatgggcctcgtgggcacctacagctgcttttggaccaagtacatgaaccacctgaccgtgcacaacagagaggtgctgtacgagctgatcgagaagagaggccctgccacccccctgatcaccgtgtccaatcaccagagctgcatggacgacccccacctgtggggcatcctgaagctgcggcacatctggaatctgaagctgatgcggtggacccccgctgccgccgatatctgcttcaccaaagagctgcacagccactttttcagcctggggaagtgcgtgcccgtgtgcagaggcgccgagttcttccaggccgagaatgagggaaagggcgtgctggacaccggcagacatatgcctggcgccggaaagcgcagagaaaagggcgacggcgtgtaccagaaaggcatggacttcatcctggaaaagctgaaccacggcgactgggtgcacatcttccccgagggcaaagtgaatatgagcagcgagttcctgcggttcaaatggggcatcggccggctgatcgccgagtgccacctgaaccctatcatcctgcccctgtggcacgtgggcatgaacgacgtgctgcccaacagccccccctacttccctagattcggccagaaaatcaccgtgctgatcggcaagcccttcagcgccctgcccgtgctggaaagactgcgggccgagaacaagagcgccgtggaaatgagaaaggccctgaccgatttcatccaggaagagttccagcacctgaaaacacaggccgagcagctgcacaaccatctgcagcccggcagatga

SEQ ID NO: 12 - protein, 292 aa, synthetic construct
MPLHVKWPFPAVPPLTWTLASSVVMGLVGTYSCFWTKYMNHLTVHNREVLYELIEKRGPATPLITVSNHQSCMDDPHLWGILKLRHIWNLKLMRWTPAAADICFTKELHSHFFSLGKCVPVCRGAEFFQAENEGKGVLDTGRHMPGAGKRREKGDGVYQKGMDFILEKLNHGDWVHIFPEGKVNMSSEFLRFKWGIGRLIAECHLNPIILPLWHVGMNDVLPNSPPYFPRFGQKITVLIGKPFSALPVLERLRAENKSAVEMRKALTDFIQEEFQHLKTQAEQLHNHLQPGR

SEQ ID NO: 13 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 14 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 15 - DNA, 789 bp, synthetic construct
  source 1..789: mol_type = other DNA
atgcccctgcacgtgaagtggcctttccctgccgtgcctcctctgacctggacactggcttccagcgtcgtgatgggcctcgtgggcacctacagctgcttttggaccaagtacatgaaccacctgaccgtgcacaacagagaggtgctgtacgagctgatcgagaagagaggccctgccacccccctgatcaccgtgtccaatcaccagagctgcatggacgacccccacctgtggggcatcctgaagctgcggcacatctggaatctgaagctgatgcggtggacccccgctgccgccgatatctgcttcaccaaagagctgcacagccactttttcagcctggggaagtgcgtgcccgtgtgcagaggcgacggcgtgtaccagaaaggcatggacttcatcctggaaaagctgaaccacggcgactgggtgcacatcttccccgagggcaaagtgaatatgagcagcgagttcctgcggttcaaatggggcatcggccggctgatcgccgagtgccacctgaaccctatcatcctgcccctgtggcacgtgggcatgaacgacgtgctgcccaacagccccccctacttccctagattcggccagaaaatcaccgtgctgatcggcaagcccttcagcgccctgcccgtgctggaaagactgcgggccgagaacaagagcgccgtggaaatgagaaaggccctgaccgatttcatccaggaagagttccagcacctgaaaacacaggccgagcagctgcacaaccatctgcagcccggcagatga

SEQ ID NO: 16 - protein, 262 aa, synthetic construct
MPLHVKWPFPAVPPLTWTLASSVVMGLVGTYSCFWTKYMNHLTVHNREVLYELIEKRGPATPLITVSNHQSCMDDPHLWGILKLRHIWNLKLMRWTPAAADICFTKELHSHFFSLGKCVPVCRGDGVYQKGMDFILEKLNHGDWVHIFPEGKVNMSSEFLRFKWGIGRLIAECHLNPIILPLWHVGMNDVLPNSPPYFPRFGQKITVLIGKPFSALPVLERLRAENKSAVEMRKALTDFIQEEFQHLKTQAEQLHNHLQPGR